(19) United States
(12) Patent Application Publication
(10) Pub. No.: US 2007/0202487 A1
(43) Pub. Date: Aug. 30, 2007

(54) Assay for phospholipidosis

(75) Inventor: Jinhong Fan, San Mateo, CA (US)

Correspondence Address: BEYER WEAVER LLP, P.O. BOX 70250, OAKLAND, CA, 94612-0250, US

(73) Assignee: Cytokinetics, Inc., A Delaware Corporation

(21) Appl. No.: 11/653,096

(22) Filed: Jan. 12, 2007

Related U.S. Application Data
(60) Provisional application No. 60/759,130, filed on Jan. 13, 2006.

(30) Foreign Application Priority Data
Mar. 8, 2006 (GB) 0604660.1

(51) Int. Cl. C12Q 1/00; G06F 19/00

(52) U.S. Cl. 435/4; 702/19

(57) ABSTRACT

Cell based assays for phospholipidosis are provided. The assays employ image and data analysis technology to provide an indication of whether a population of cells exhibits phospholipidosis. Methods to assess the effect of a stimulus on inducing phospholipidosis in a population of cells are also provided.

### CROSS-REFERENCE TO RELATED PATENT APPLICATIONS

This application claims priority under 35 U.S.C. §119(e) to U.S. provisional application No. 60/759,130, filed on Jan. 13, 2006 and titled ASSAY FOR PHOSPHOLIPIDOSIS, incorporated herein by reference for all purposes. This application also claims priority under 35 U.S.C. §119 to Great Britain application No. 0604660.1, filed Mar. 8, 2006 and also titled ASSAY FOR PHOSPHOLIPIDOSIS, incorporated herein by reference for all purposes. This application is related to US Patent Publication No. US 2005-0014217 A1 of Mattheakis et al., published Jan. 20, 2005, and titled “PREDICTING HEPATOTOXICITY USING CELL BASED ASSAYS,” and to US Patent Publication No. US 2005-0014216 of Mattheakis et al., published Jan. 20, 2005, and titled “PREDICTING HEPATOTOXICITY CELL BASED ASSAYS,” both of which are incorporated herein by reference for all purposes.

Provide are methods and apparatus for assessing whether a population of cells exhibits phospholipidosis. Provided are image analysis methods and apparatus that determine whether a population of cells exhibit phospholipidosis based on the phenotypic characteristics of the cells.

Hepatotoxicity is a major safety concern for drug development. Approximately 90 percent of lead candidates fail to become drugs, and hepatotoxicity accounts for about 22 percent of these failures. One hepatotoxic pathology that can be induced by drugs is phospholipidosis. Phospholipidosis is a disorder characterized by an excessive build-up of phospholipids in cells. In addition to other effects, phospholipidosis affects lysosome function. Lysosomes are subcellular organelles necessary for digestion of extracellular molecules, damaged or old cell parts and microorganisms. Lysosomes play an important role in detoxification of waste products.

Traditionally, a variety of strategies have been used to predict hepatotoxicity during preclinical development. These include incubating compounds with cultured hepatocytes to measure cytotoxicity or induction of the various isoforms comprising the drug metabolizing CYP enzymes. Biochemical enzyme assays, using purified CYP enzymes or crude liver microsome extracts, are used to determine the substrate activities of drug candidates and to profile their metabolic products using chromatographic methods. Animal studies have also been widely used to predict human hepatotoxicity. In these studies, rats or mice are dosed with various concentrations of the test compound, and the animals are monitored for important serum markers such as serum albumin, prothrombin, bilirubin, AST, ALT, and alkaline phosphate at different time points. The animals are then sacrificed, and a full histopathological analysis of the liver, kidney, and other important organs and/or tissues is carried out.

Provided are methods and apparatus to assess the effect of a stimulus on inducing phospholipidosis in a population of cells. In certain embodiments, imaging technologies are used to analyze the effects of a stimulus on hepatocytes or other cell types. Also provided are methods to determine if a population of cells exhibits phospholipidosis.

Certain embodiments provide methods of determining whether a population of cells exhibits phospholipidosis by performing the following operations: (a) contacting the population of cells with a lysosomal marker, (b) imaging the population of cells, (c) analyzing images of the population of cells to determine information about lysosomes in the cells, and (d) determining whether the population of cells exhibits phospholipidosis based on the information.

Certain embodiments provide methods of assessing the hepatotoxicity of a stimulus, by performing the following operations: (a) exposing a population of hepatocyte cells to the stimulus, (b) contacting the population of cells with a lysosomal marker, (c) imaging the population of cells, (d) analyzing images of the population to determine information about lysosomes in the cells, and (e) characterizing the phospholipidotic response of the population of cells to the stimulus based on the information.

Certain embodiments provide computer program products including machine-readable media on which are stored program instructions for implementing a portion of or an entire method as described above. Any of the methods described herein may be represented, in whole or in part, as program instructions that can be provided on such computer readable media.

These and other features and advantages will be described in more detail below with reference to the associated figures.

### BRIEF DESCRIPTION OF THE DRAWINGS

FIG. 1 is an electron microscope image of mouse liver cells exhibiting phospholopidosis.

FIG. 2A shows Hoechst and LysoTracker® images of hepatocyte cells cultured in DMSO.

FIG. 2B shows LysoTracker® images of hepatocyte cells treated with five compounds as compared to DMSO control.

FIG. 3 is a flow chart depicting various operations performed in determining whether a cell population exhibits phospholipidosis according to certain embodiments.

FIG. 4 is a flow chart depicting various operations performed in assessing the hepatotoxicity of a stimulus according to certain embodiments.

FIGS. 5A and 5B show dose response curves for chlorpromazine as generated by a method according to certain embodiments presented herein.

FIG. 6 is a diagrammatic representation of a computer system that can be used with methods and apparatus described herein.

One hepatotoxic pathology is phospholipidosis, a disorder that affects lipid storage. Triglyceride lipids accumulate in the cells, including in lysosomes. Lysosomes are cellular organelles that perform controlled degradation of macromolecules. Degradative enzymes in an acidic pH (˜pH 5) in the interior of the lysosome are used to digest intra and extracellular debris and phagocytosed microorganisms. A lysosomal membrane protects the cytosol from these enzymes and stabilizes the pH in and out of the lysosome. Transport proteins in the membrane pump ions from the cytosol to the lysosome interior to regulate the pH.

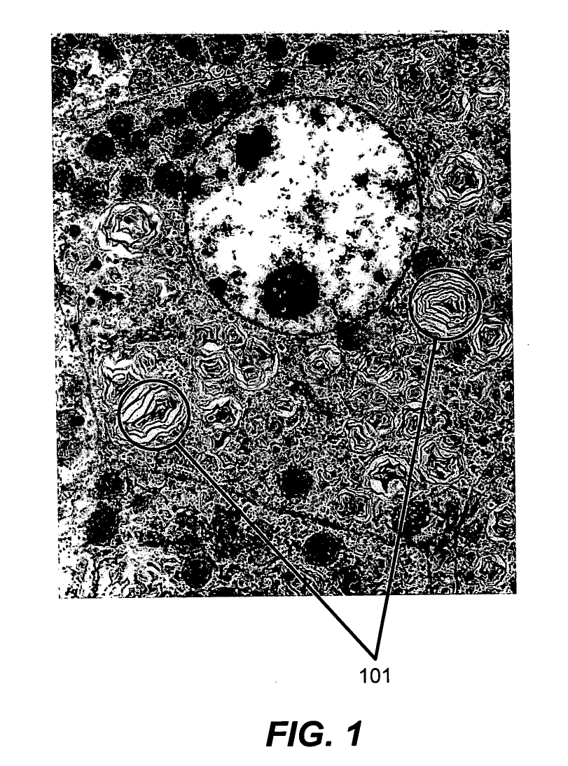

Excessive phospholipid accumulation in the lysosomes is related to the formation of onion-like multi-layered structures associated with lysosomes. These multi-layered vesicles structures are sometime referred to as Mallory bodies. FIG. 1 shows an electron microscope image of a mouse liver dosed with a phospholipidotic compound. Reference number 101 indicates lysosome derived Mallory bodies typical of phospholipidosis. The formation of the lysosome derived Mallory bodies in hepatotocytes indicates that phospholipidosis affects lysosome function.

Image and data analysis technology can be used to provide an indication of whether a population of cells exhibits phospholipidosis in an in vitro cell culture system. Specifically, in certain embodiments, whether a population exhibits phospholipidosis based on phenotypic characteristics of the cells is determined. These characteristics are derived in whole or in part from analyzing a cell image showing the positions and concentrations of one or more markers bound within the cells. In certain embodiments, the methods provide an indication of whether a population of cells exhibits phospholipidosis without the use of electron microscopy.

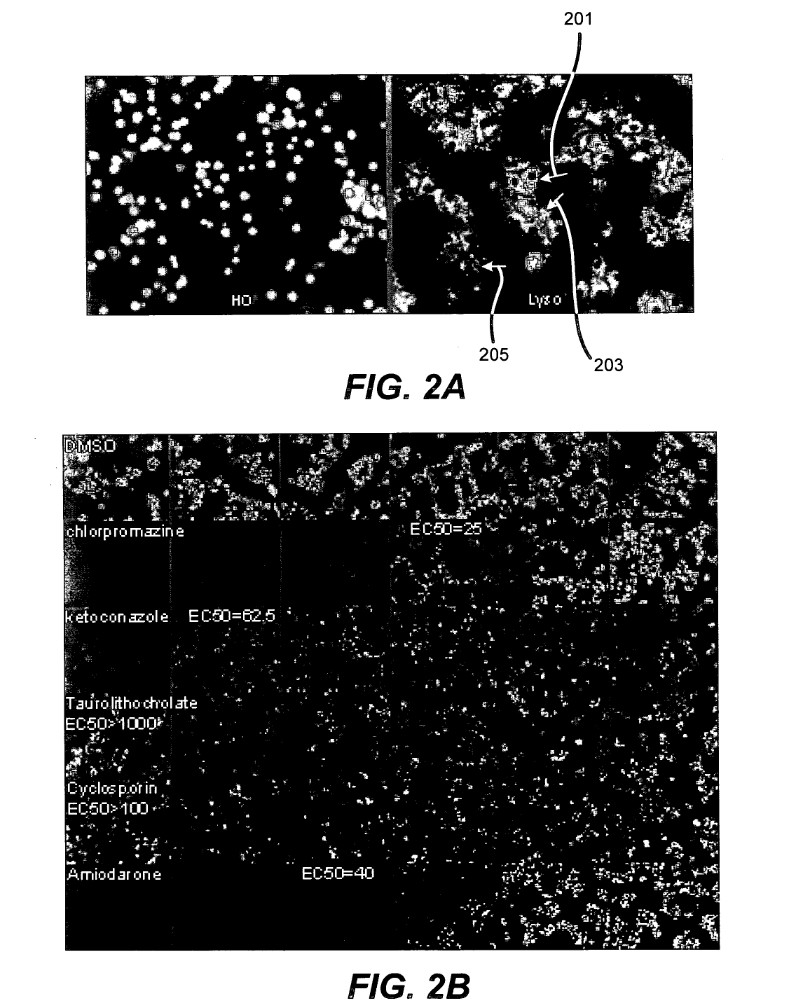

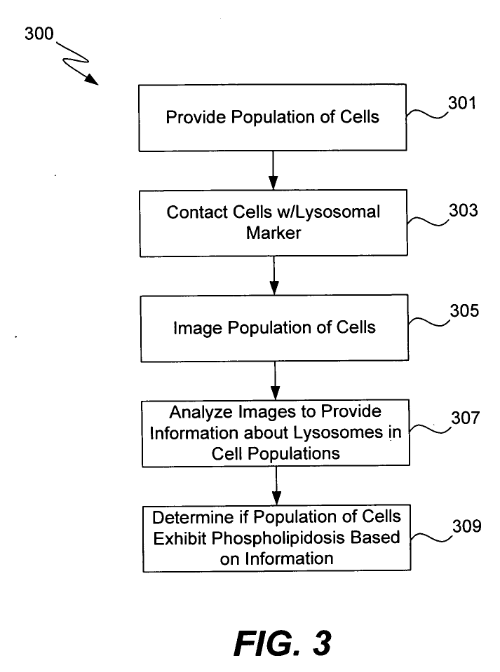

FIG. 2A shows hepatocyte cells in DMSO stained with Hoechst 33341, a DNA stain, and LysoTracker®, a lysosomal stain. The Hoechst image, showing DNA within the population of cells, is on the left. Each of the bright spots in this image is an area of high DNA concentration, i.e., a nucleus. Images of the lysosomes in the population, as stained by the LysoTracker® dye, are on the right. Lysosomes appear as perinuclear (reference numbers 201 and 203) or pericanalicular structures (reference number 205). The lysosome structures shown in FIG. 2 may also be classified by intensity (brightness) and the amount of “punctate” staining. A punctate structure is a structure wherein small holes interrupt a flat distribution. In the lysosome image in FIG. 2, lysosome 201 appears as a bright/punctate structure, whereas lysosome 203 appears as dim/smooth structure.

Phospholipidosis is not limited to hepatotocytes, but occurs in a wide range of systems (e.g., cell types, cell lines, tissues, etc.) other than hepatocyte systems. For convenience, the description provided herein refers primarily to hepatotocytes.

Phospholipidosis may be induced by various stimuli. One class of compounds that induce phospholipidosis are Cationic Amphiphic Drugs (CAD) compounds. Many, though not all, CAD compounds induce phospholipidosis. Phospholipidosis is also induced by non-CAD compounds. Compounds that induce phospholipidosis are generally referred to as phospholipidotic compounds. Examples of phospholipidotic compounds include chlorpromazine and amiodarone.

FIG. 2B shows LysoTracker® images of cells treated with 5 compounds at six concentrations and six DMSO controls. Hepatocytes were treated with various compounds that induce phospholipidosis and/or choleostasis, specifically chlorpromazine, ketocanazole, taurolithocholate, cyclosporin and amiodarone. As indicated, chlorpromazine and amiodarone induce phospholipidosis. Ketocanazole, taurolithocholate and cyclosporine induce choleostasis. (In some cases, some of the compounds induce more than one pathology; specifically, ketacanazole may induce phospholipidosis, chlorpromazine may induce choleostasis and amiodarone may induce steatosis). The EC50 of cell death for each compound is shown in the figure. Compound concentration decreases from left to right; the left panel shows the highest compound concentration. As can be seen by looking at the panels on the right of FIG. 2B, at several concentrations below EC50 the phospholipidotic compounds chlorpromazine and aniodarone increase the proportion of bright/punctate cells. Taurolithocholate and cyclosporine decrease the proportion of bright/punctate cells. All compounds induce a significant reduction in LysoTracker® staining intensity for all cells at concentrations below the EC50. As described further below, in certain embodiments, the number of bright and/or punctate regions in the lysosomal marker image is used as an indication of phospholipidosis.

In addition to the number or proportion of lysosomes that appear as bright and/or punctate structures, phospholipidosis may also induce an increase in the number of lysosomes that are pericanalicular (as opposed to perinuclear). Thus, in certain embodiments, the location of the lysosomes as indicated by the lysosomal marker is used an indication of phospholipidosis.

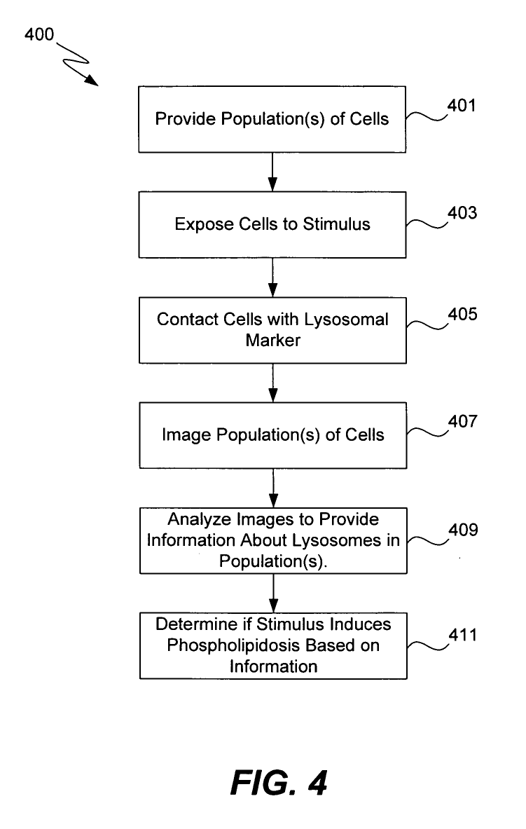

FIG. 3 is a process flow sheet that depicts important operations of a method of determining whether a population of cells exhibits phospholipidosis according to certain embodiments. Process 300 begins when a population of cells is provided at block 301. In certain embodiments, the cells are hepatocytes derived from rats, humans, or other species. Techniques for preparing hepatocyte cultures are discussed below in Section II: Culturing Hepatocytes for Assay. At block 303 the population is contacted with a lysosomal marker. As used herein, a lysosomal marker is a marker that selectively binds to, accumulates in, or otherwise selectively marks lysosomes. Examples of lysosomal markers are given below in Section III: Markers. Next, the population of cells is then imaged at block 305. Single or multiple images may be acquired from the same well. Frequently, multiple images will be taken at different “channels.” Each such channel may be associated with a different marker, which highlights a specific feature of the cell such as a particular macromolecule or organelle. An example is shown in FIG. 2 wherein the left image shows an image of the DNA maker Hoechst 33341 and the right image shows an image of the lysosomal marker LysoTracker®. Each separate image provides a different piece of information that can be used to characterize the hepatocytes in culture. In the process depicted in FIG. 3, at least one channel is associated with a lysosomal marker.

The process continues at block 307 in which the images are analyzed to provide information about the lysosomes in the population of cells. Although not depicted in the flow sheet, in certain embodiments, the individual cells of the image are separately analyzed, apart from the background in the image, to obtain statistical results that characterize the impact of the stimulus on various features of the cells. To this end, the image must be “segmented” to separate regions of the image associated with individual cells. Each “segment” of the image is a group of contiguous pixels associated with an individual cell. Whether the population of cells is segmented or not, the image or images analyzed to provide information about the lysosomes in the population. For instance, information about phenotypic characteristics such as the intensity, distribution, location and morphology of the lysosomes may be provided. Further discussion of the information that may be provided by the image analysis is described below in Section IV: Analyzing Images.

This information obtained from the images is then used to determine if the population of cells exhibits phospholipidosis in block 309. In certain embodiments, information about other phenotypic characteristics is used in addition the information about the lysosomes to determine if the cells exhibit phospholipidosis. This determination may be made in some embodiments by applying the information to a model. Using the image analysis information to determine whether the cells exhibit phospholipidosis is discussed further in Section V: Using Image Analysis Information to Predict Phospholipidosis.

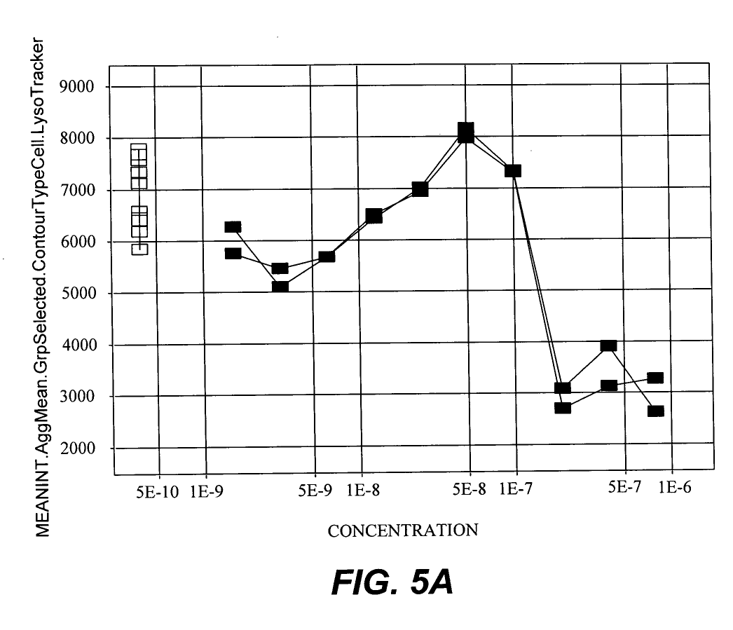

Certain embodiments provide methods that assess whether a particular compound or other stimulus induces phospholipidosis. FIG. 4 shows a process flow sheet depicting operations according to certain embodiments of these methods. As depicted, process 400 begins when a population (or populations) of cells is provided in block 401. The cells are exposed to the stimulus under investigation in block 403. Many different stimuli may be tested. In a typical example, hepatocytes in multiple wells are contacted with a test compound. The compound may be provided at different concentrations in different wells. In at least one well, a control well, no compound is used. In another case, the multiple wells may be contacted with the compound for different lengths of time prior to fixing. These approaches allow a generation of a stimulus response path. Each concentration or time point provides a different phenotypic point on the response path. The cells are contacted with a lysosomal marker in block 405.

The next block in the depicted process (block 407) involves obtaining an image or images of the hepatocytes exposed to the stimulus under investigation. The images are then analyzed to provide information about the lysosomes in the population(s) of cells in block 409. Blocks 405-409 are performed as described with reference to the corresponding steps (303-307) in FIG. 3. The information is then used to determine if the stimulus should be classified as inducing phospholipidosis in block 411. Determining whether the stimulus induces phospholipidosis may involve generating a stimulus response path. This is discussed further in Section V: Using Image Analysis Information to Predict Phospholipidosis.

Determining whether a stimulus induces phospholipidosis may be performed alone or as part of a broader assay to determine the hepatotoxicity of a stimulus. For example, U.S. Patent Publication No. US-2005-0014217-A1 titled PREDICTING HEPATOTOXICITY USING CELL BASED ASSAYS, hereby incorporated by reference for all purposes, describes cell based assays to predict hepatotoxicity of stimuli including determining whether stimuli induce pathologies such as apoptosis, necrosis, cholestasis, and/or steatosis. One of skill in the art will understand given the description provided how the phospholipidosis assays described herein may be incorporated into the assays described in U.S. Patent Publication No. US-2005-0014217-A1.

Some of the terms used herein are not commonly used in the art. Other terms may have multiple meanings in the art. Therefore, the following definitions are provided as an aid to understanding the description herein. The claims should not necessarily be limited by these definitions.

The term “component” or “component of a cell” refers to a part of a cell having some interesting property that can be characterized by image analysis to derive biologically relevant information. General examples of cell components include biomolecules and subcellular organelles. Specific examples of biomolecules that could serve as cell components include proteins and peptides, lipids, polysaccharides, nucleic acids, etc. Sometimes, the relevant component will include a group of structurally or functionally related biomolecules. Alternatively, the component may represent a portion of a biomolecule such as a polysaccharide group on a protein, or a particular subsequence of a nucleic acid or protein. Collections of molecules such as micells can also serve as cellular components. And subcellular structures such as vesicles and organelles may also serve the purpose.

The term “cell population” is used interchangeably with “population of cells.” A population of cells may include one or more cells. In certain embodiments, a population of cells is the cells in a well on a plate and referred to as a well. In certain embodiments, a population of cells is the cells in a field of view taken from an image of cells in a well or other support medium.

The term “marker” or “labeling agent” refers to materials that specifically bind to and label cell components. These markers or labeling agents should be detectable in an image of the relevant cells. Typically, a labeling agent emits a signal whose intensity is related to the concentration of the cell component to which the agent binds and the signal intensity is directly proportional to the concentration of the underlying cell component. The location of the signal source (i.e., the position of the marker) should be detectable in an image of the relevant cells.

The term “stimulus” refers to something that may influence the biological condition of a cell. Often the term will be synonymous with “agent” or “manipulation.” Stimuli may be materials, radiation (including all manner of electromagnetic and particle radiation), forces (including mechanical (e.g., gravitational), electrical, magnetic, and nuclear), fields, thermal energy, and the like. General examples of materials that may be used as stimuli include organic and inorganic chemical compounds, biological materials such as nucleic acids, carbohydrates, proteins and peptides, lipids, various infectious agents, mixtures of the foregoing, and the like. Other general examples of stimuli include non-ambient temperature, non-ambient pressure, acoustic energy, electromagnetic radiation of all frequencies, the lack of a particular material (e.g., the lack of oxygen as in ischemia), temporal factors, etc.

An important class of stimuli is chemical compounds, including compounds that are drugs or drug candidates and compounds that are present in the environment. The biological impact, including toxicity, of chemical compounds is frequently manifest as clear phenotypic changes.

Specific examples of biological stimuli include exposure to drug candidate compounds, hormones, growth factors, antibodies, or extracellular matrix components. Or exposure to biologics such as infective materials such as viruses that may be naturally occurring viruses or viruses engineered to express exogenous genes at various levels. Biological stimuli could also include delivery of antisense polynucleotides by means such as gene transfection.

Other specific stimuli include exposure of cells to conditions that promote cell fusion. Specific physical stimuli could include exposing cells to shear stress under different rates of fluid flow, exposure of cells to different temperatures, exposure of cells to vacuum or positive pressure, or exposure of cells to sonication. Another stimulus includes applying centrifugal force. Other specific stimuli include changes in gravitational force, including sub-gravitation, application of a constant or pulsed electrical current. Still other stimuli include photobleaching, which in some embodiments may include prior addition of a substance that would specifically mark areas to be photobleached by subsequent light exposure. In addition, these types of stimuli may be varied as to time of exposure, or cells could be subjected to multiple stimuli in various combinations and orders of addition. Of course, the type of manipulation used depends upon the research endeavor at hand.

The term “phenotype” generally refers to the total appearance of an organism or cell from an organism. cellular phenotypes and their representations in processing systems (e.g., computers) are interesting. The phenotypic characteristics of a cell are functions of the cell's genetic constitution and environment. Often a particular phenotype can be correlated or associated with a particular biological condition or mechanism of action resulting from exposure to a stimulus. Generally, cells undergoing a change in biological conditions will undergo a corresponding change in phenotype. Thus, cellular phenotypic data and characterizations may be exploited to deduce mechanisms of action and other aspects of cellular responses to various stimuli.

A selected collection of data and characterizations that represent a phenotype of a given cell or group of cells is sometimes referred to as a “quantitative cellular phenotype.” This combination is also sometimes referred to as a phenotypic fingerprint or just “fingerprint.” The multiple cellular attributes or features of the quantitative phenotype can be collectively stored and/or indexed, numerically or otherwise. The attributes are typically quantified in the context of specific cellular components or markers. Measured attributes useful for characterizing an associated phenotype include morphological descriptors (e.g., size, shape, and/or location of the organelle) and composition (e.g., concentration distribution of particular biomolecules within the organelle). Other attributes include changes in a migration pattern, a growth rate, cord formation, an extracellular matrix deposition, and even cell count. Often, the attributes represent the collective value of a feature over some or all cells in an image (e.g., some or all cells in a specific well of a plate). The collective value may be an average over all cells, a mean value, a maximum value, a minimum value or some other statistical representation of the values.

The quantitative phenotypes may themselves serve as individual points on “response curves.” A phenotypic response to stimulus may be determined by exposing various cell lines to a stimulus of interest at various levels (e.g., doses of radiation or concentrations of a compound). In each level within this range, the phenotypic descriptors of interest are measured to generate quantitative phenotypes associated with levels of stimulus.

The term “path” or “response curve” refers to the characterization of a stimulus at various levels. For example, the path may characterize the effect of a chemical applied at various concentrations or the effect of electromagnetic radiation provided to cells at various levels of intensity or the effect of depriving a cell of various levels of a nutrient. Mathematically, the path is made up of multiple points, each at a different level of the stimulus. Each of these points (sometimes called signatures) may be a collection of parameters or characterizations describing some aspect of a cell or collection of cells. Typically, at least some of these parameters and/or characterizations are derived from images of the cells. In this regard, they represent quantitative phenotypes of the cells. In the sense that each point or signature in the path may contain more than one piece of information about a cell, the points may be viewed as arrays, vectors, matrices, etc. To the extent that the path connects points containing phenotypic information (separate quantitative phenotypes), the path itself may be viewed as a “concentration-independent phenotype.” The generation and use of stimulus response paths are described in more detail in U.S. patent application Ser. No. 09/789,595, filed Feb. 20, 2001 naming Vaisberg et al., and titled, “CHARACTERIZING BIOLOGICAL STIMULI BY RESPONSE CURVES,” and U.S. patent application Ser. No. 10/623,485, filed on Jul. 18, 2003, naming V. Kutsyy, D. Coleman, and E. Vaisberg as inventors, and titled, “Characterizing Biological Stimuli by Response Curves,” both of which are incorporated herein by reference for all purposes.

As used herein, the term “feature” refers to a phenotypic property of a cell or population of cells. Typically, the points in a response curve are each comprised of multiple features. The terms “descriptor” and “attribute” may be used synonymously with “feature.” Features derived from cell images include both the basic “features” extracted from a cell image and the “biological characterizations” (including biological classifications such as cell cycle states). The latter example of a feature is typically obtained from an algorithm that acts on a more basic feature. The basic features are typically morphological, concentration, and/or statistical values obtained by analyzing a cell image showing the positions and concentrations of one or more markers bound within the cells.

### II. Culturing Hepatocytes for Assay

Hepatocyte cultures are used in assays designed to assess hepatotoxicity. Some are used as controls and others are exposed to one or more stimuli that may produce toxic responses in hepatocytes. As explained, the cultures are imaged and analyzed to identify features that may be affected by the stimuli tested. The features are analyzed in order to categorize the stimulus according to pathology (or simply toxicity), as will be described in more detail below.

Hepatocyte cultures may be derived from rats, humans or other species appropriate to the stimulus under investigation. Generally, hepatocytes used in different experiments should give consistent responses when exposed to the same assay conditions. In certain embodiments, they come from a relatively homogeneous pool so that cells taken from one source respond similarly to cells taken from a different source. Laboratory rats, being relatively homogeneous genetically in comparison to most human groups, may provide a suitably consistent source of hepatocytes for assays. However, the effects of a stimulus on rat hepatocytes sometimes fail to adequately represent the effects seen on human hepatocytes. Hence, human hepatocytes may be necessary for some investigations.

Transformed or immortalized human hepatocyte cell lines can provide a genetically homogeneous source for many assays. One widely used transformed hepatocyte cell line is HepG2 available from the American Type Culture Center as HB 8065. Hep G2 is also available from Amphioxus Cell Technologies of Houston, Tex.

Unfortunately, immortalized cells do not always provide a completely realistic model of normal hepatocytes. In particular, although the hepatoma derived cell lines are easy to culture and maintain, they may not express the full complement of Cytochrome P450 metabolizing enzymes. One approach to this problem is to genetically modify the immortalized cells to mimic the expression pattern of a non-immortalized cell.

For non-immortalized or primary cells, one may use either freshly-isolated cells, which have been recently harvested, or cryopreserved cells. Several commercial vendors provide fresh or cryopreserved primary hepatocytes from human, rat, dog, and primate species. These vendors include Xenotech LLC of Lenexa, Tex.; Tissue Transformation Technologies of Edison, N.J.; In Vitro Technologies of Baltimore, Md.; Gentest (a BD Biosciences company) of Woburn, Mass.; and BD Biosciences. Transplant ready/fresh human hepatocytes are available from In Vitro Technologies and Tissue Transformation Technologies. Freshly isolated cells are normally used within 24 hrs to 96 hrs after harvesting.

Although non-immortalized cells generally present a better model for hepatotoxicity in assays than their immortalized counterparts, immortalized cells are usually easier to use. As indicated, primary human hepatocytes have a finite shelf-life and exhibit significant genetic variation across samples. Accordingly, another approach to culturing hepatocytes for an assay can include isolating a highly differentiated hepatocyte cell line that retains the metabolic activity of primary hepatocytes but has been immortalized to allow easy cultivation in vitro. For a more detailed description of established techniques for preparing such cell lines, see Kobayashi, et al., “Prevention of acute liver failure in rats with reversibly immortalized human hepatocytes,” Science, 287:1258-1262 (2000).

Hepatocyte cultures can be grown in or on various support structures. For instance, a bare plastic support that includes nutrients can be used to support a culture. Similarly, a glass surface can be used to support a culture. Other kinds of supports can include extra-cellular matrices such as collagen or Matrigel (available from BD Biosciences, San Jose, Calif.). Such structures can be provided in multiwell plates, such as 384-well assay plates. Cultures of primary hepatocytes generally grow well in three-dimensional lattice structures.

In some embodiments, hepatocytes can be cultured with associated cells to encourage the hepatocytes to behave naturally in an assay. For instance, hepatocytes can be co-cultured with stromal cells such as fibroblasts. Co-culturing hepatocytes and support cells in this manner may improve the predictive qualities of the assays in some contexts.

A discussion of co-culturing is provided in U.S. Pat. No. 6,599,694 of Elias (issued Jul. 29, 2003), which is incorporated herein by reference for all purposes. As explained there, two separate cell types are exposed to a stimulus suspected of producing a biological condition (e.g., a specific toxic pathology). The two different cell types are co-cultured or otherwise allowed to interact with one another before and during exposure to the agent. The images of the cells show how the stimulus separately affects each of the cell types. Specifically, the images show how the phenotype of each cell type changes (or does not change) upon exposure to the stimulus. In this regard, the concept of a phenotype encompasses visual indicators showing migration patterns, growth rates, extracellular matrix depositions, etc.

The cultures and supports described above may provide in vitro models of in vivo hepatocyte functioning. For example, a three-dimensional co-culture of primary human liver stroma and parenchymal cells can be provided in vitro in a manner that mimics in vivo liver tissue function.

For some assays, such as the cholestasis assay, it may be appropriate to culture “polarized hepatocytes.” Such culture can mimic features of the biliary tree, such as bile ducts, and thereby cause the hepatocytes to secrete bile into a “duct” (the exposed portion of the culture) and otherwise behave as if they were part the biliary tree. Because cholestasis is characterized by inhibition of bile flow, such culturing facilitates characterization of cholestasis.

An exemplary procedure for preparing hepatocyte cultures will now be described. Specifically, the procedure involves the use of primary rat hepatocytes as follows:

Isolation and culture of primary rat hepatocytes

1. Adult Sprague-Dawley male rats (250-350 g) are anesthetized with Isoflurane to induce an anesthetic plane by inhalation.

2. An initial incision is made with surgical scissors to the ribcage, proximal to the pubis through the skin and muscle wall, with care taken to avoid cutting the diaphragm.

3. The intestines are then moved from the abdominal cavity to the animal's side. The portal vein is exposed and two surgical sutures are loosely placed around the portal vein.

4. The peristaltic pump is turned on and the flow rate is set to 1 mL/min using Liver Perfusion Medium warmed to 37° C. (available from Gibco BRL, Div. of Life Technologies Inc., Gaithersburg, Md., Catalog # 17701).

5. The portal vein is cannulated and the portal vein sutures are tightened. With care taken to avoid introducing any air bubbles, the perfusion line is connected to the catheter.

6. The animal is euthanized by cutting the heart and diaphragm. The rate of the peristaltic pump is slowly brought to 25 mL/min and perfused for 10 minutes.

7. After 10 minutes, the perfusion media is switched to Liver Digest Medium (Gibco BRL, Div. of Life Technologies Inc., Gaithersburg, Md., Catalog # 17703) at 37° C. and perfused for 10 minutes.

8. The liver is removed and placed in a 10 cm dish which contains ice cold wash medium (Invitrogen, cat # 17704). Next, the surface capsule tissues are torn open with a fine pair of forcepts and cells were released from liver by gently swirling the liver in the cold medium. A total of 200 ml of medium containing cells are collected and filtered through sterile 100 micron cell strainer and divided into 4 50 ml comical tubes.

9. The contents are centrifuged for 5 minutes at 50 g. The supernatant is then removed and the pellet is resuspended in fresh cold wash medium. This step of centrifugation and resuspension is repeated 4 times. The pellet after final spin is suspended in plating medium (Willium E Medium supplemented with FCS).

10. The total cell count and vialibity are determined by Trypan blue exclusion.

11. Twenty three thousand cells/well are plated in a 96 flat well plate pre-coated with collagen I (BD Biosciences, San Jose, Calif. or CellzDirect, Tucson, Ariz.). The plates are incubated at 37° C./5% CO₂ for two hours. At the end of the two hour incubation, plating medium is aspirated and cold Hepatozyme medium containing 250 micrograms/ml of Matrigel (BD Biosciences) is added, and the plates are incubated overnight.

As appreciated by those of skill in the art, other procedures for preparing hepatocyte cultures can also be used.

### II. Lysosomal Markers

As used herein, a lysosomal marker is a marker that selectively binds to, accumulates in, or otherwise selectively marks lysosomes. The marker typically binds specifically to lysosomes (or a subset of lysosomes) regardless of location within the cell. The marker should provide a strong contrast to other features in a given image. To this end, the marker should be luminescent, radioactive, fluorescent, etc. Various stains and compounds may serve this purpose. Examples of such compounds include fluorescently labeled antibodies to the cellular component of interest, fluorescent intercalators, and fluorescent lectins. The antibodies may be fluorescently labeled either directly or indirectly.

An example of a marker that is used according to some embodiments is LysoTracker®, available from Invitrogen. LysoTracker® molecular probes are fluorescently labeled weak bases that accumulate in low pH environments. LysoTracker® is membrane permeable and accumulates in lysosomes, which as indicated above, have a pH of about 5. Other appropriate markers that selectively stain lysosomes may also be used.

### III. Imaging and Segmentation

As indicated, the phenotypic data characterizing the cell populations and/or stimuli is derived, at least in part, from images of hepatocytes, which are in some embodiments exposed to particular combinations of stimulus type and stimulus level. Sees block 305 in FIG. 3 and block 407 in FIG. 1B, for example. Various techniques for preparing and imaging appropriately treated cells are described in the following U.S. patent applications: Ser. No. 09/310,879 by Crompton et al., entitled A DATABASE METHOD FOR PREDICITIVE CELLULAR BIONINFORMATICS, filed on May 14, 1999; U.S. Pat. No. 6,651,008 by Crompton et al., issued Nov. 18, 2003; and U.S. Pat. No. 6,631,331 by Crompton et al., issued Oct. 7, 2003, each of which are incorporated by reference herein for all purposes.

Generally the images used are obtained from cells that have been specially treated and/or imaged under conditions that contrast the cell's marked components with other cellular components and the background of the image. Typically, the cells are fixed, optionally washed, and then treated with a material that binds to the components of interest and shows up in an image (i.e., the marker). In certain embodiments the chosen agent specifically binds to the cellular component of interest, but not to most other cellular biomolecules. In some cases, the cells are treated with the marker prior to fixation.

In the case of cells treated with a fluorescent marker, a collection of such cells is illuminated with light at an excitation frequency. A detector is tuned to collect light at an emission frequency. The collected light is used to generate an image, which highlights regions of high marker concentration.

Additional operations may be performed prior to, during, or after the imaging operation. For example, “quality control algorithms” may be employed to discard image data based on, for example, poor exposure, focus failures, foreign objects, and other imaging failures. Generally, problem images can be identified by abnormal intensities and/or spatial statistics.

In a specific embodiment, a correction algorithm may be applied prior to segmentation to correct for changing light conditions, positions of wells, etc. In one example, a noise reduction technique such as median filtering is employed. Then a correction for spatial differences in intensity may be employed. In one example, the spatial correction comprises a separate model for each image (or group of images). These models may be generated by separately summing or averaging all pixel values in the x-direction for each value of y and then separately summing or averaging all pixel values in the y direction for each value of x. In this manner, a parabolic set of correction values is generated for the image or images under consideration. Applying the correction values to the image adjusts for optical system non-linearities, mis-positioning of wells during imaging, etc.

Note that the quality of the images is dependent on cell plating, compound dilution, compound addition and imaging focusing. Failures in any these systems can be detected by a variety of methods. For example, cell plating could fail because of a clogged tip in a delivery pipette. Such failure can be identified by adding a fluorescent dye or bead to the cell suspension. The fluorescence of this dye or bead is chosen to be at a different channel (wavelength) than the markers used to image cellular components. Another potential failure could occur during compound delivery. To detect such failures, one can add a fluorescent dye or bead in the compound plate before compound dilution. The amount of fluorescent dye or bead is proportional to the amount of compound. Yet another potential problem occurs when the focus of the image acquisition system changes during imaging. To account for such spatial biases, one can employ control wells containing, for example, cells with no or neutral compounds interspersed throughout the plate. Still another problem results from foreign objects (e.g., small dust particles) in the well. This can be addressed with image segmentation and statistical outlier identification techniques. Both manual and automated methods can be used to eliminate bad images from analysis.

Growing cells on a three-dimensional matrix such as collagen or Matrigel may also present some challenges for imaging. In particular, autofocusing can be difficult when cells are located in a three-dimensional structure. However, culturing conditions and automated microscopy capabilities can be adjusted to keep a sufficient number of cells within an accessible focal plane. Furthermore, the spatial resolution can be adjusted according to the degree of magnification necessary for a particular assay. Other conditions that can be modified according to the segmentation needs of a particular assay include the use of markers (e.g., DAPI for DNA) and the cell fixation procedures implemented. An example of an automated microscopy system is the ImageXpress available from Molecular Devices or the Discovery 1 available from Universal Imaging and Molecular Devices.

The goal of segmentation is to allow feature extraction on a cell-by-cell basis. Segmentation identifies discrete regions of an image that include only those pixels where the components of a single cell are deemed to be present. Thus, each representation is a bounded collection of pixels, each providing associated features characterizing a single cell.

Segmentation can be accomplished in numerous ways. These include use of techniques that identify regions of high DNA concentration (presumed to be nuclear regions) and watershed algorithms. Typically nuclear DNA markers provide a strong signal and there is a high contrast in the image and an edge detection based segmentation process can be used. The segmentation process typically identifies edges where there is a sudden change in intensity of the cells in the image and then looks for closed connected edges in order to identify an object. In some cases, segmentation can be conducted on confluent or semi-confluent cultures

In one approach to segmentation, the image analysis tool initially identifies the nucleus of each cell captured in the image under consideration. If images from different channels are well registered, the nuclei can be first identified in the DNA channel and then overlaid to the image under consideration. The segmentation algorithm defines a “ring region” around each nucleus. Generally, this step serves to define the perinuclear region. This region encompasses some or all of the cytoplasmic cell components in a normal interphase cell. The general method is described in US Published Patent Application No. US-2002-0141631-A1, published Oct. 3, 2002, naming Vaisberg, Cong, and Wu as inventors, and titled “IMAGE ANALYSIS OF THE GOLGI COMPLEX,” which is incorporated herein by reference for all purposes.

To identify bi-nuclear cells (and not treat each nuclei as the locus of different cell), one may employ nearest neighbor algorithms and other algorithms of the type described in U.S. patent application Ser. No. 10/615,116, filed Jul. 7, 2003, naming Coleman et al., and titled “METHODS AND APPARATUS FOR CHARACTERISING CELLS AND TREATMENTS,” which is incorporated herein by reference for all purposes.

A watershed algorithm has been found to provide very good segmentation even in cultures containing many abutting hepatocytes or cells of other types. A suitable algorithm for this purpose is described in US Published Patent Application No. US-2002-0154798-A1, published Oct. 24, 2002, naming Cong and Vaisberg as inventors and, titled “EXTRACTING SHAPE INFORMATION CONTAINED IN CELL IMAGES.” which is incorporated herein by reference for all purposes. The watershed approach does a good job of correctly illustrating the shape of cells identified during segmentation. In some embodiments, it employs image data for a cell shape-indicative marker (for example, cytoskeletal components, (e.g., tubulin), one or more cytoplasmic proteins (for example lactate dehydrogenase or total cell protein), or membrane components (e.g., lipids or plasma membrane receptors) in a watershed technique. Markers that detect proteins localized on the cell surface may work well in this technique. Examples include the tight junction proteins zonula occludens-1 (ZO-1), ZO-2, and ZO-3, which are found at the interface of fused hepatocytes and other cells. Other reagents for segmenting cells include succinimidyl esters conjugated to fluorescent dyes such as TAMRA or Alexa (Molecular Probes, Eugene, Oreg.). This reagent labels the primary amine groups of proteins and can be used to label any cells, including hepatocytes.

The following example is a procedure for fixing and staining primary rat hepatocytes to visualize nuclei, lysotracker, and Alexa 647 nm succinimidyl ester reagent (A647SE). A647SE is used to segment individual hepatocytes within an image.
1) At 24 hr after compound addition to cells, 50 ul of Hepatozyme medium (prewarmed to 37° C.) containing 187.5 nM of lysotracker dye is added to each well. Plates are placed back to incubator for 30 min, then 150 μl of fix solution (8% paraformaldehyde, in 1× PBS) and incubate at room temperature for 30 min.
2) Aspirate the fix solution, and add 350 μl of wash buffer (1× PBS, 0.02% Triton X-100) to each well. Aspirate and dispense wash buffer 2 times.
3) Aspirate off the wash buffer, and add 100 ul per well of A647SE mix: 0.5 μg/ml A647SE (stock 10 mg/ml) and 1:1000 dilution of Hoechst 33342 (stock 5 mg/ml) in wash buffer. Incubate at room temperature for one hour in the dark.
4) Aspirate the staining solution, and add 350 μl of wash buffer (1× PBS, 0.2% Triton X-100) to each well. Aspirate and dispense wash buffer 2 times. Leave the last wash buffer in the well.
5) Seal the plate and store at room temperature in the dark prior to imaging.

### IV. Image Analysis

As indicated, in certain embodiments, images of the cells are analyzed to provide information about the lysosomes in the cell population. See block 307 in FIG. 3 and block 409 in FIG. 4, for example. This information or phenotypic data is then used to determine if the population exhibits phospholipidosis and/or a stimulus of interest induces phospholipidosis. See block 309 in FIG. 3 and block 411 in FIG. 4, for example.

Referring back to FIG. 2A, the lysosomes may appear as bright (high marker intensity, e.g., lysosome 201) or dim (low marker intensity, e.g., lysosome 203). Additionally the lysosomes appear as punctate structures (i.e., there are holes in the distribution of marker intensity as for lysosome 201) or smooth structures (i.e., the distribution of marker intensity is even as for lysosome 203).

As discussed above in reference to FIG. 3, phospholipidosis is associated with an increase in the number of lysosomes appearing as bright and/or punctate structures. This is believed to be related to the formation of Mallory bodies also associated with phospholipidosis. Phospholipidosis may also induce an increase of lysosomes that are pericanalicular rather than perinuclear.

Thus, in certain embodiments, image analysis provides information about the morphology, distribution (e.g. punctate or smooth) and location of the lysosomes. In certain embodiments, phenotypic characteristics associated with phospholipidosis that may be derived from image analysis include the amount or relative amount of lysosomes that appear as bright and/or punctate structures, as well as the location of lysosomes within the cell.

These phenotypic characteristics may be characterized by various features obtainable from the images. These include the following: features associated with granularity, total or average intensity of the marker, standard deviation (or variance) of the intensity of the marker and/or higher order moments of the intensity of the marker including kurtosis and skewedness.

Granularity refers to bright spots or granules representing intercellular organelles or other objects in images. Because the bright/punctate structures (such as lysosome 201 in FIG. 2) appear as bright spots or granules, granularity analysis may be used to characterize the amount of bright and/or punctate structures. In certain embodiments, granularity algorithms that detect and quantify objects that are substantially smaller than cells are used. Such algorithms may take advantage of the fact that granules in an image of a population of cells are located at those places in the image where an edge can be found close to a local intensity maximum. Thus, by combining an edge detection analysis of an image with a local intensity detection analysis of the image, the granules can be located and quantified within the image.

In various embodiments, the extracted features can include, for example, the number of granules, the total surface area of the granules and the mean or maximum intensities of the granules. Extracting these and other features associated with granularity from an image is described in U.S. Provisional Patent Application No. 60/757,597, filed Jan. 9, 2006 (Atty. Docket No. CYTOP160P) and in U.S. patent application Ser. No. ______, filed Jan. 9, 2007 (Atty. Docket No. CYTOP160), both titled GRANULARITY ANALYSIS IN CELLULAR PHENOTYPES, which are hereby incorporated by reference for all purposes. In certain embodiments, features associated with granularity are determined on a per cell basis. In certain embodiments, features associated with granularity are determined per cell-region basis. For example, the number of granules in a periphery region of the cell may be determined. A discussion of determining features in a cell periphery is described in U.S. Provisional Patent Application No. 60/757,598, filed Jan. 9, 2006 (Atty. Docket No. CYTOP159P) and in U.S. patent application Ser. No. ______, filed Jan. 9, 2007 (Atty. Docket No. CYTOP159), both titled DOMAIN SEGMENTATION AND ANALYSIS, which are hereby incorporated by reference for all purposes. Determining features in a periphery region may be useful to characterize the number or amount of lysosomes in pericanalicular versus perinuclear regions.

As indicated, phospholipidosis is associated with an increase in the amount of bright structures. Thus, the total and/or mean intensity of the lysosomal marker may be used to provide an indication of phospholipidosis. The total and mean intensity may be calculated on a per cell, per granule or per cell-region basis.

Phospholipidosis is also associated with an increase in the amount of punctate structures. Thus lysosome distribution (punctate or smooth) may provide an indication of phospholipidosis. Lysosome distribution may be characterized by the total or mean intensity of pixel intensity, standard deviation or variance of the lysosome marker pixel values, one or more moments of pixel intensity, kurtosis or skewedness of pixel intensity or granularity. These features may be calculated on a per cell, per granule or per cell-region basis. As described above, features associated with granularity may also be used to characterize lysosome distribution.

Also as indicated, phospholipidosis may be associated with an increase in the number or proportion of lysosomes are pericanalicular rather than perinuclear. This may be characterized by determining any of the above features (e.g., granularity, intensity or moments) in whole cells, cell periphery regions and perinuclear reagions. In certain embodiments, the values of these features derived from different domains may be compared against each other by computing their ratios, and the ratio value is used to indicate the re-distribution of lysosomes induced by phospholipidosis.

These features described above may be used alone or in combination with other features to characterize the population of cells. For example, in some embodiments, linear or non-linear combinations of features may be used.

### V. Using Image Analysis Information to Predict Phospholipidosis

As indicated, incertain embodiments, the information about lysosomes obtained from image analysis may be used to determine if a population of cells exhibits phospholipidosis. This may be done using a variety of modeling methodologies known to those skilled in the art. In certain embodiments, the model may provide a binary classification (yes/no) of whether the population exhibits phospholipidosis. In other embodiments, the model may provide a number indicating a predictive score or severity of the pathology. Also as indicated, the information about the lysosomes obtained from image analysis may be used to determine if a stimulus induces phospholipidosis. This prediction may also be binary or non-binary.

The models may be mixture models, decision trees, linear expressions, non-linear expressions, etc. For example, in certain embodiments, a mixture model may be used to determine if the population of cells exhibit phospholipidosis. Mixture models as applied to other types of classifications of cells are described in U.S. Patent Publication No. 20050272073 titled PLOIDY CLASSIFICATION METHOD and U.S. patent application Ser. No. 11/082,241, filed Mar. 15, 2005 and titled ASSAY FOR DISTINGUISHING LIVE AND DEAD CELLS. In some embodiments, a random forest model may be used. Random forest models are described in U.S. Application No. 60/758,733, filed Jan. 13, 2006 (Atty. Docket No. CYTOP161P) and in U.S. application No. ______, filed concurrently with the present application (Atty. Docket No. CYTOP161), both titled RANDOM FOREST MODELING OF CELLULAR PHENOTYPES. Each of these references is hereby incorporated by reference for all purposes.

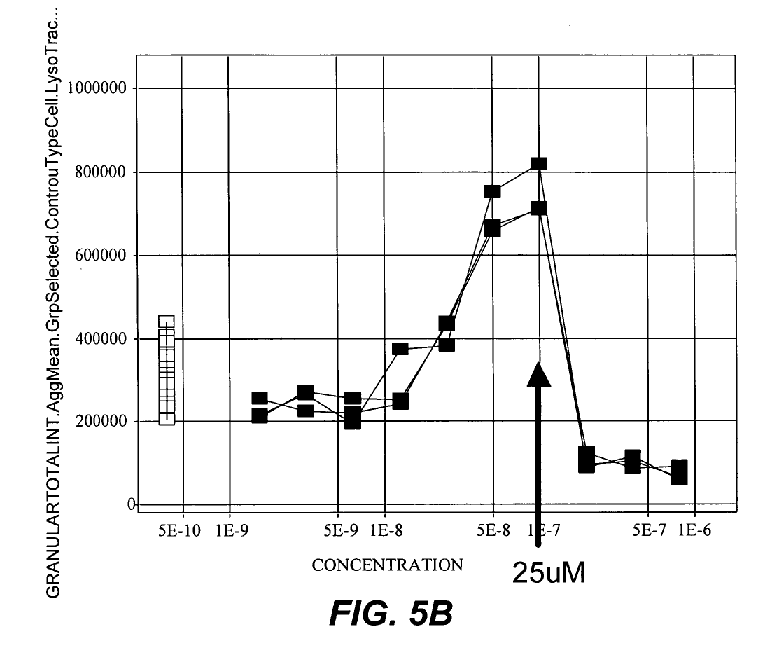

In certain embodiments, a stimulus response curve may be generated for a stimulus of interest. In certain embodiments, the stimulus response curve plots the feature or features used to characterize phospholipidosis (e.g., granularity or mean intensity) against concentrations of a stimulus. FIGS. 5A and 5B show examples of stimulus response curves for chlorpromazine. In FIG. 5A, the mean intensity of the LysoTracker® marker (as measured on a per-cell basis) is plotted against concentration. The arrow indicates the IC50 of cell death is 25 μM. As can be seen from the response curve, chlorpromazine increases mean LysoTracker® intensity, causing a spike prior to the IC50 concentration. This indicates that the compound induced phospholipidosis. FIG. 5B shows total granularity plotted against concentration. The curve spikes at concentration lower than the IC50 of cell death, also indicating that the compound induced phospholipidosis. Similar dose response curves may be generated for any particular stimulus to determine if the compound induces phospholipidosis.

In certain embodiments, models to determine if a stimulus induces phospholipidosis may be generated using a variety of methodologies known to those of skill in the art. U.S. patent application Ser. No. 10/623,485 (“Characterizing Biological Stimuli by Response Curves,” filed on Jul. 18, 2003), incorporated by reference above, describes one such technique. It identifies relevant features and other parameters for image analysis classification models by analyzing stimulus response paths for various known toxins or other stimuli. The response paths and known mechanisms of action (or pathologies) comprise a training set for the model. Various potential models are compared based on their ability to correctly classify members of the training set. The classification is accomplished using distance measurements between the various stimulus response paths in a multi-dimensional feature space. A discussion employing such methods to generate hepatotoxicity models is discussed in US Patent Publication No. US 20050014216, incorporated by reference above. Similar methods may be employed to generate phospholipidosis models.

To use such the model one extracts the relevant features (e.g., granularity, mean intensity) from an image of hepatocytes treated with a stimulus having unknown toxicity. Then one measures distances between these features from untreated cells and features obtained from cells treated with other stimuli having known toxic responses. Classification is based on distance (in feature space) between the features of the test stimuli and features of various pre-classified control stimuli. Alternatively, one can use a regression technique, a neural network, a support vector machine, etc. To develop an expression or analytical tool that takes feature values as input values and calculates a classification (pathology).

### VI. Software/Hardware

Generally, methods described above may employ various processes involving data stored in or transferred through one or more computer systems. Also provided is an apparatus or apparatuses for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, also provided are computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). Data and program instructions may also be embodied on a carrier wave or other transport medium. Examples of program instructions include both machine code, such as produced by a compiler, and files containing higher level code that may be executed by the computer using an interpreter.

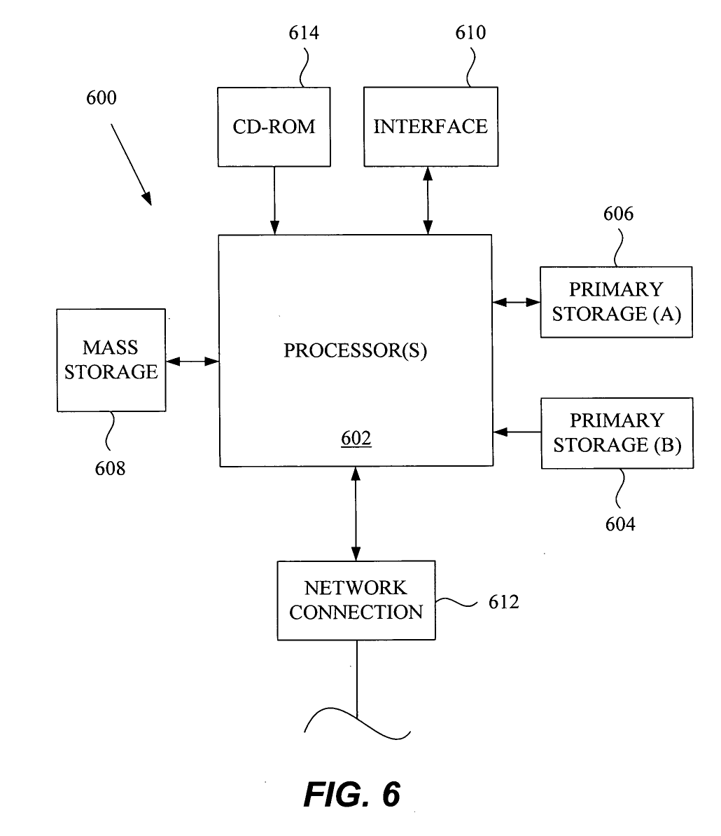

FIG. 6 illustrates a typical computer system that, when appropriately configured or designed, can serve as an image analysis apparatus. The computer system 600 includes any number of processors 602 (also referred to as central processing units, or CPUs) that are coupled to storage devices including primary storage 606 (typically a random access memory, or RAM), primary storage 604 (typically a read only memory, or ROM). CPU 602 may be of various types including microcontrollers and microprocessors such as programmable devices (e.g., CPLDs and FPGAs) and unprogrammable devices such as gate array ASICs or general purpose microprocessors. As is well known in the art, primary storage 604 acts to transfer data and instructions uni-directionally to the CPU and primary storage 606 is used typically to transfer data and instructions in a bi-directional manner. Both of these primary storage devices may include any suitable computer-readable media such as those described above. A mass storage device 608 is also coupled bi-directionally to CPU 602 and provides additional data storage capacity and may include any of the computer-readable media described above. Mass storage device 608 may be used to store programs, data and the like and is typically a secondary storage medium such as a hard disk. It will be appreciated that the information retained within the mass storage device 608, may, in appropriate cases, be incorporated in standard fashion as part of primary storage 606 as virtual memory. A specific mass storage device such as a CD-ROM 614 may also pass data uni-directionally to the CPU.

CPU 602 is also coupled to an interface 610 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 602 optionally may be coupled to an external device such as a database or a computer or telecommunications network using an external connection as shown generally at 612. With such a connection, it is contemplated that the CPU might receive information from the network, or might output information to the network in the course of performing the method steps described herein.

In one embodiment, the computer system 600 is directly coupled to an image acquisition system such as an optical imaging system that captures images of cells. Digital images from the image generating system are provided via interface 612 for image analysis by system 600. Alternatively, the images processed by system 600 are provided from an image storage source such as a database or other repository of cell images. Again, the images are provided via interface 612. Once in the image analysis apparatus 600, a memory device such as primary storage 606 or mass storage 608 buffers or stores, at least temporarily, digital images of the cell. With this data, the image analysis apparatus 600 can perform various image analysis operations. To this end, the processor may perform various operations on the stored digital image. For example, it may analyze said image in manner that extracts values of one or more descriptors and classifies as exhibiting phospholipidosis.

### VII. Other Embodiments

The above discussion has focused on hepatocytes and hepatotoxic responses. Phospholipidosis is not limited to hepatotocytes, but occurs in a wide range of systems (e.g., cell types, cell lines, tissues, etc.) other than hepatocyte systems. Thus, the description provided herein extends beyond hepatotoxicity to toxicity in a variety of other cell lines, cell types, and tissues.

Although the above generally describes specific exemplary processes and apparatus, various modifications can be made without departing from the spirit and/or scope of the description provided.

What is claimed is:

1. A method of determining whether a population of cells exhibits phospholipidosis, the method comprising:
(a) contacting the population of cells with a lysosomal marker;
(b) imaging the population of cells;
(c) analyzing one or more images of the population of cells to determine information about lysosomes in the cells; and
(d) determining whether the population of cells exhibits phospholipidosis based on the information.

2. The method of claim 1 wherein (c) comprises determining information about at least one of the morphology, distribution and location of the lysosomes in the cells or region s of the cells.

3. The method of claim 1 wherein (c) comprises determining information about the granularity, intensity or distribution of the lysosomal marker in the cells or regions of the cells.

4. The method of claim 3 wherein (c) comprises determining the mean intensity of the marker in the cells.

5. The method of claim 3 wherein (c) comprises determining the total intensity of the marker in the cells.

6. The method of claim 3 wherein (c) comprises determining the total granularity of the marker in the cells.

7. The method of claim 3 wherein (c) comprises determining the mean granularity of the marker in the cells.

8. The method of claim 3 wherein (c) comprises determining kurtosis of the intensity of the marker in the cells.

9. The method of claim 1 wherein (c) comprises determining information about the amount of lysosomes that appear punctate and/or bright.

10. The method of claim 1 wherein (d) comprises applying the information to a mixture model, one or more decision trees, or a linear or non-linear expression.

11. A method of assessing the hepatotoxicity of a stimulus, the method comprising:
(a) exposing a population of hepatocyte cells to the stimulus;
(b) contacting the population of cells with a lysosomal marker;
(c) imaging the population of cells;
(d) analyzing one or more images of the population to determine information about lysosomes in the cells; and
(e) characterizing the phoshopholipidotic response of the population of cells to the stimulus based on the information.

12. The method of claim 11 wherein (d) comprises determining information about at least one of the morphology, distribution and location of the lysosomes in the cells or regions of the cells.

13. The method of claim 11 wherein (d) comprises determining information about the granularity, intensity or distribution of the lysosomal marker in the cells or regions of the cells.

14. The method of claim 11 wherein (d) comprises determining the mean intensity of the marker in the cells.

15. The method of claim 11 wherein (d) comprises determining the total intensity of the marker in the cells.

16. The method of claim 11 wherein (d) comprises determining the total granularity of the marker in the cells.

17. The method of claim 11 wherein (d) comprises determining the mean granularity of the marker in the cells.

18. The method of claim 11 wherein (d) comprises determining kurtosis of the intensity of the marker in the cells.

19. The method of claim 11 further comprising repeating steps (a)-(d) for multiple concentrations of the stimulus and (e) comprises generating a dose response curve.

20. The method of claim 11 wherein (d) comprises determining the difference between the information determined in (c) and information determined for pre-classified stimuli.

21. A computer program product comprising a machine readable medium on which is provided program instructions for determining whether a population of cells exhibits phospholipidosis, the program instructions comprising:
(a) code for analyzing images of the population to determine information about lysosomes in the cells, wherein the images comprise a signal corresponding to a lysosomal marker within the cells; and
(b) code for determining whether the population of cells exhibits phospholipidosis based on the information.

22. The computer program product of claim 21 wherein (a) comprises code for determining information about the granularity, intensity or distribution of the lysosomal marker in the cells or regions of the cells.

23. A computer program product comprising a machine readable medium on which is provided program instructions for assessing the hepatotoxicity of a stimulus, the program instructions comprising:
(a) code for analyzing images of a population of cells exposed to the stimulus to determine information about lysosomes in the cells, wherein the images comprise a signal corresponding to a lysosomal marker within the cells; and
(f) code for characterizing the phoshopholipidotic response of the population of cells to the stimulus based on the information.

24. The computer program product of claim 21 wherein (a) comprises code for determining information about the granularity, intensity or distribution of the lysosomal marker in the cells or regions of the cells.
